(19) United States
(12) Patent Application Publication
(10) Pub. No.: US 2004/0024043 A1
(43) Pub. Date: Feb. 5, 2004

(54) Method for treating cognitive disorders

(76) Inventors: Nigel Greig, Phoenix, MD (US); Gosse Bruinsma, Leiden (NL)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/386,915

(22) Filed: Mar. 12, 2003

Related U.S. Application Data
(60) Provisional application No. 60/367,068, filed on Mar. 22, 2002.

(51) Int. Cl. A61K 31/407

(52) U.S. Cl. 514/411

(57) ABSTRACT

Compositions and methods for the treatment of diseases resulting from cognitive Disorders, such as Alzheimer's diseases with the compound (−) N-(−)-N phenyl canbamoyleseroline as the active ingredient.

### CROSS REFERENCE TO RELATED APPLICATIONS

This Application claims priority of U.S. Provisional Serial No. 60/367,068 filed Mar. 22, 2002, incorporated herein by reference.

### TECHNICAL FIELD

The present invention relates to methods for the treatment of diseases resulting from cognitive disorders, such as Alzheimer's disease to ameliorate the affects which and slow down the progression of these diseases.

### BACKGROUND OF INVENTION

In the past, the compounds useful for treating cognitive disorders, such as Alzheimer's disease, have included donepezil, rivastigmine and galanthamine based upon their activity, as set forth in U.S. Pat. No. 5,409,948, Apr. 25, 1995,as acetylcholinesterase inhibitors. In addition, phenserine, the negative optical enantiomer (−) N-(−)-N phenyl canbamoyleseroline, which has the structure

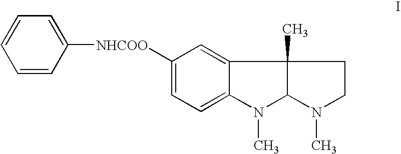

and its salts,

another acetylcholinesterase inhibitor is being used clinically for treating cognitive disorders.

Due to the fact that these compounds are all anticholinesterase inhibitors they have serious drawbacks producing undesirable side affects caused by their activity as acetylcholinesterase inhibitors. These undesirable side effects are related to their toxicity caused by their suppression of acetylcholinesterase. Due to the fact that these compounds, which are administered chronically, have a low therapeutic ratio (i.e. the ratio between toxicity and therapeutic effect) they produce a number of pathologic conditions associated with cholinergic under activity. Therefore due to the chronic nature of treatment for cognitive disorders it has long been desired to provide an agent which is effective and does not produce the toxic side effects inherent in the use of acetylcholinesterase inhibitors.

### SUMMARY OF INVENTION

In accordance with this invention, it has been found that the compound of the formula

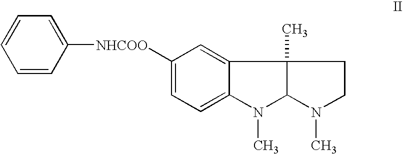

or their pharmaceutically acceptable salts,

can be used to treat patients having cognitive disorders such as Alzheimer's disease and cognitive impairments associated with aging without the side effects caused by the toxic profile of anticholinesterase inhibitors

This invention is directed to a method of treating patients with cognitive disorders by orally administering the compound of formula II or its pharmaceutically acceptable salts and compositions for administering the compound to patients.

### BRIEF DESCRIPTION OF DRAWINGS

Reference is now made accompanying this application wherein:

FIG. 1 illustrates that phenserine reduces secreted and cellular βAPP levels in a concentration dependent manner.

FIG. 2 illustrates that phenserine's action on βAPP translates into reduced Aβ protein levels.

FIG. 3 demonstrates that the positive isomer of phenserine reduces the production of βAPP and Aβ protein in the same manner as phenserine.

### DETAILED DESCRIPTION

In accordance with this invention, it has been found that the compound of formula II and its pharmaceutically acceptable salts are effective for treating patients suffering from cognitive disorders and can be administered orally to patients without the toxic side effects caused by anticholinesterase activity associated with such compound phenserine, rivastigmine, donepezil and galanthamine. This is especially surprising in view of the fact that the compound of the formula II, which is (+) 9-N-phenylcarbinol esroline is the non natural (+) isomer of phenserine, the compound of formula I and has minimal anticholinesterase activity. In fact unlike phenserine, the Compound of Formula I and its salts have very little, if not any, anticholinesterase inhibition activity. Therefore toxic effects such as nausea, vomiting, dizziness tremor, bradycardia, etc, caused when anticholinesterase agents are administered, are not seen utilizing the method of this invention In accordance with this invention, it has been discovered that the (+) enantiomer of phenserine is a potent inhibitor of the progression of cognitive impairment associated with aging or Alzheimer's disease. The compound of formula II has been disclosed by Pei, Greig, et al. Article entitled “Inhibition of Human Acetylcholinesterase” Med Cem Resarch Acad. (1995) 5: 265-270. In this article, it was shown that unlike its negative enantiomer phenserine, the compound of the formula II was far less active as an inhibitor of human acetylcholinesterase. However, despite this, it has been found in accordance with this invention, that the compound of formula II is potent in the reduction of the levels of the potentially toxic amyloid-β peptide (Aβ) and that this Aβ protein reduces a progressive neurodegenerative condition leading to loss of memory characterized by the appearance of senile plaques that are primarily composed of an Aβ and neurofibrillary tangle aggregates. The Aβ is a 40- to 42-residue peptide derived from a larger protein βAPP, a protein which contains 695-770 residues. βAPP is converted into the Aβ protein which can produce the pathological hallmarks of cognitive impairments.

As part of this invention it has been found that the compound of formula II and its pharmaceutically acceptable salts, like phenserine can manipulate the βAPP protein to produce nonamyloidogenic byproducts and thereby reduce the production of the Aβ protein. In view of the fact that the compound of formula II, unlike its negative enantiomer phenserine, is not a potent anticholinesterase inhibitor, it does not produce the side effects caused by anticholinesterase inhibition activity. That the (+) enantiomeric form is not very potent inhibitor of acetylcholinesterase can be seen from the results reported in the Shaw, et al. publication Proc. Natl. Academy Science USA (2001) 98 (13, 76057610) where it is stated, “The concentration of compound required to inhibit 50% acetylcholinesterase activity was 22 nM for (−)-phenserine, whereas >25,000 nM was inactive for (+)-phenserine.” Therefore by the procedure and results disclosed in the Shaw, et al. publication, unlike the negative enantiomer of phenserine, the compound of formula II and its salts are not effective inhibitors for acetylcholinesterase.

In accordance with this invention, the (+) enantiomer of formula II is effective for the treatment of Alzheimer's disease, minimal cognitive impairment in age-associated memory impairment including any other dementia associated with cognitive impairment. In addition, unlike use of the other therapeutic agents for treating cognitive impairment, the compound of formula II and its salts due to the fact that they lack anticholinesterase activity are more effective and do not have the toxic side effects associated with anticholinesterase inhibitors such as nausea, diarrhea, vomiting, dizziness and bradycardia. That the compound of formula II and/or its salts do not affect cholinesterase allows the compounds of this invention to be administered to patients at high dosage levels to achieve good results in treatment without the danger of the toxic side effects.

The method of treatment of this invention is directed to patients having a disease state which exhibits the cognitive impairments and symptoms associated with aging or Alzheimer's disease. In may of the patients suffering from such cognitive impairment, it is difficult to definitively diagnose whether these symptoms are directly attributable to Alzheimer's disease or the aging process. Therefore the method of this invention is applicable to patients especially those patients over 50 years old who are suffering from a disease state which exhibits the cognitive impairment symptoms associated with aging or Alzheimer's disease.

The dosage for treatment typically depends upon the route of administration, the age, weight and condition with regard to cognitive impairment of the patient to be treated. In general, dosages of from 0.5 mg. to 10 mg. per kg. per day compound of formula II and/or its salt given orally to the patient produce the beneficial effects. In accordance with this invention, it is generally preferred to utilize oral dosages of from 1.0 mg/kg to 5.0 mg/kg per day, with dosages of from 1 mg. per kg. to 2 mg. per kg. per day being especially preferred. The compound of formula II and/or its salts can be administered orally from 1 to 4 times a day at the dosage levels given above. It is important to note that any treatment for cognitive disorders such as Alzheimer's disease and other age-related cognitive impairments require chronic treatment (i.e. that is continuous treatment) throughout the life of the patient. In this manner, the deterioration due to cognitive impairment from these cognitive disorders and the symptoms of such cognitive impairment are stabilized or ameliorated and in some cases improved. The cognitive disorders which result from such diseases are progressive throughout the life of the patient. Through the treatment of this invention, prevents the progression of these cognitive disorders. Therefore, the method of this invention provides a means for reducing the progression of these disease states.

The ability of the compound of formula II and/or its salt to improve cognitive performance can be assessed by various known means. Among these means are the standard tests for measuring the progression of this disease state such as the Mini, Mental State Examination and the Clinical Dementia Rating as well as the Alzheimer's Disease Assessment Scale (ADAS-cog). The ADAS-cog is a multi-item instrument for measuring cognitive performance which include elements of memory, orientation, retention, reasoning, language and praxis. The ADAS-cog scoring ranges from 0 to 70 with higher scores indicating cognitive impairment. Elderly normal adults may score as low as 0 to 1, but it is not unusual for non demented adults to score more highly. In measuring by the ADAS-cog test, one measures the changes over extended period of time before and during treatment to determine the progression of this disease and also to compare this rating with untreated patients. In patients treated in accordance with the method of this invention it is found that during treatment those patients treated have the same or better scores under this test as compared to untreated patients. Also the ability of the method of this invention to produce overall results clinically, can be assessed using the Clinical Interview Board Impression Of Change (CIBIC test). This test takes the results from caregivers as well as of physicians who interview the patients and test the patient functions such as their general cognitive functions, behavioral functions and their activities of daily living. The CIBIC score plus is scored as a 7 point categorical rating ranging from a score of 1 indicating markedly improved to a score of 4 indicating no change to a score of 7 indicating markedly worse. During treatment in accordance with this invention most patients receive scores of 4 and some receive better scores (i.e. lower scores). On the other hand, with respect to non treated patients having the same assessment at the same given time most of the patients received higher scores (i.e. greater than 4), which indicated a worsening of their condition.

The compound of formula II is produced by (+) esroline via the following reaction scheme

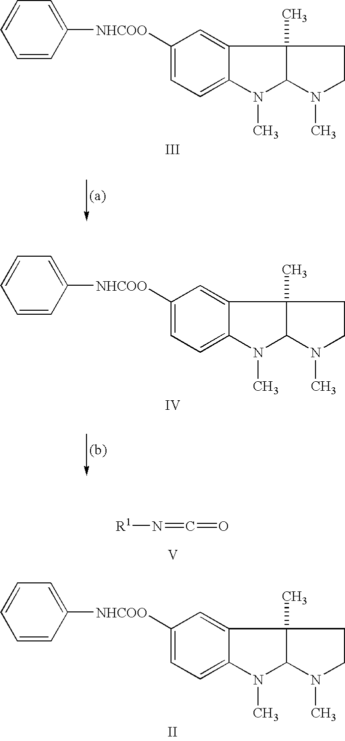

wherein R¹ is phenyl.

In accordance with the process of this invention the physostigmine compound of Formula III or it's salt are reacted to form the (−) eseroline compound of Formula IV by hydrolyzing the physostigmine compound of Formula III with an alkali metal hydroxide, in an aqueous reaction medium. The eseroline compound of Formula IV is then isolated in pure form, from the aqueous reaction medium.

The purified eseroline is then treated with a strong organic base in an anhydrous reaction medium containing a water miscible organic solvent. The treated eseroline compound is then reacted, without isolating it from the said reaction medium, with an isocyanate of the formula V. This reaction is carried out by mixing said isocyanate compound of formula V with said eseroline compound in said reaction medium to form said enantiomer of formula II. Thereafter the reaction is quenched by addition of water allowing (+) phenserine compound of formula III to be easily isolated in pure form. In this addition, the water can be added to the reaction mixture or the reaction mixture can be added to water. Generally it is preferred to add the reaction mixture to water.

In accordance with this invention any pharmaceutically acceptable acid addition salt of the compound of Formula II can be used in the treatment method and compositions of this invention. The term “pharmaceutically acceptable salts” refers to acid addition salts. The expression “pharmaceutically acceptable acid addition salts” is intended to apply to any non-toxic organic or inorganic acid addition salt of the compound of Formula II, with the preferred salt being a tartrate salt. Illustrative inorganic acids which form suitable salts include hydrochloric, hydrobromic, sulfuric, and phosphoric acid and acid metal salts such as sodium monohydrogen orthophosphate, and potassium hydrogen sulfate. Illustrative organic acids which form suitable salts include the mono-, di-, and tricarboxylic acids. Illustrative of such acids are, for example, acetic, glycolic, lactic, pyruvic, malonic, succinic, glutaric, fumaric, malic, tartaric, citric, ascorbic, maleic, hyroxymaleic, benzoic, hydrocybenzoic, pheynlacetic, cinnamic, salicylic, 2-phyenoxybenzoic, and sulfonic acids such as p-toluenesulfonic acid, methanesulfonic acid and 2-hydroxyethanesulfonic acid.

In accordance with this invention, the aforementioned compound or formula I or its pharmaceutically acceptable salts are useful in pharmaceutically acceptable oral or transdermal administration with oral administration being preferred. These pharmaceutical compositions of the invention for oral or transdermal administration contain said compound for formula I or its pharmaceutically acceptable salts in association with a compatible pharmaceutically acceptable carrier material. Any conventional carrier material can be utilized. The carrier material can be an organic or inorganic inert carrier material suitable for such administration. Suitable carriers include water, gelatin, gum arabic, lactose, starch, magnesium stearate, talc, vegetable oils, polyalkylene-glycols, petroleum jelly and the like. Furthermore, the pharmaceutical preparations may contain other pharmaceutically active agents. Additional additives such as flavoring agents, preservatives, stabilizers, emulsifying agents, buffers and the like may be added in accordance with accepted practices of pharmaceutical compounding.

The compound of formula II and/or its pharmaceutically acceptable salts can be administered in accordance with the preferred embodiment of this invention in an oral unit dosage form. Any of the above conventional oral unit dosage forms can be utilized with the preferred unit dosage form being tablets or capsules. The daily dose for achieving the desired affect can be obtained by utilizing oral unit dosage forms containing from about 20 to 300 mg. of active ingredient with oral unit dosage forms containing from about 50 to 150 mg. being especially preferred. Besides the carriers these oral dosage forms generally contain conventional recipients such as binder, disintegrates, lubricants and glydants. In addition, any of the conventional methods utilized formulating these oral unit dosage forms can be utilized in accordance with this invention.

The pharmaceutical preparations can be made up in any conventional oral unit dosage form including a solid form for oral administration such as tablets, capsules, pills, powders, granules, and the like. The pharmaceutical preparations may be sterilized and/or may contain adjuvants such as preservatives, stabilizers, wetting agents, emulsifiers, salts for carrying the osmotic pressure and/or buffers.

The invention is further illustrated by the following examples which are only for illustrative purposes and not eliminative thereof.

### EXAMPLES

### Example 1

Under an argon atmosphere, a 50 wt % sodium hydroxide solution (67.7 g, 0.8462 mol) is added dropwise to a slurry of the (+) enantiomer of physostigmine salicylate (100 g, 0.2418 mol) in degassed DI water (300 mL) at 45° C. During the addition the temperature is kept between 45 and 55° C. After about 3 hours at 45° C. the yellow solution is cooled to 25 to 30° C. and tert.-butyl methyl ether (300 mL) is added. The pH of the aqueous phase is adjusted to 9.1 with an aqueous solution of sodium meta bisulfite (54 g, Na₂S₂O₅, 250 mL water). The mixture is stirred for 30 minutes, the phases are allowed to settle and then separated. The aqueous phase is extracted twice for 30 minutes each with tert.-butyl methyl ether (300 mL each). The organic phases were combined and washed three times with 20 wt % sodium chloride solution (200 mL each), then they are dried over magnesium sulfate (150 g) overnight. The slurry is filtered through Celite and the filter cake washed with tert.-butyl methyl ether. The filtrate was concentrated to 300 mL at 25 to 29 in of vacuum and the residue co-distilled twice with diethoxymethane (300 mL each). The residue is diluted with diethoxymethane (300 mL) and heated to 50° C. The obtained light slurry is cooled to 5° C., stirred for 45 minutes, then concentrated to about 300 mL. Cold heptane (300 mL) is added dropwise, the slurry is stirred for 20 minutes and the volume increased by addition of cold heptane (125 mL). After stirring for about 2 hours the slurry is filtered via a Buchner funnel. The collected solid is washed with cold heptane (200 mL) then dried in vacuo overnight. The (+) eseroline base (35.6 g) is obtained as a white solid in 67.4% yield and 98.3% purity.

### Example 2

The (+) eseroline enantiomer (50 g, 0.229 mol) is dissolved in 400 mL anhydrous dimethoxyethane under an argon atmosphere. Catalytic amounts Of 2.5 M n-butyl lithium in hexanes (6.4 mL, 16 mmol) are added within 1 minute and the solution stirred for 10 minutes. Phenyl isocyanate (27.269 g, 0.2286 mmol) is added over 32 minutes keeping the temperature between 20 and 23° C. The reaction solution is stirred at r.t. for 2 hours 20 minutes, then transferred to an addition funnel. The reaction solution is added over 49 minutes to mixture of DI water (630 mL) and dimethoxyethane (42 mL) under vigorous stirring. The obtained slurry is stirred for 30 minutes, then it is filtered via a Buchner funnel (Whatman #3 filterpaper). The solid residue is washed four times with DI water (100 mL each) and once with heptane (100 mL), then it is dried at 45° C. and >29 inches of vacuum for 9 hours. The (+) enantiomer of N-phenyl carbonamoyl eseroline (74.4 g) is obtained as reddish solid in 96.2% yield and 95.1% purity.

### Example 3

Under an argon atmosphere a solution of tartaric acid (17.12 g, 0.114 mol) in a mixture of anhydrous ethanol (131 mL) and DI water (3.3 mL) is added over 32 minutes to a slurry of the (+) enantiomer of N-phenyl carbanoyl eseroline prepared above (35 g, 0.1037 mol) in a mixture of anhydrous ethanol (126 mL) and DI water (3.1 mL). After about 60 to 75% of the tartaric acid solution were added the reaction solution is seeded with phenserine tartrate (72 mg). The reaction mixture is stirred for 19 hours 15 minutes at room temperature then a mixture of isopropanol (490 mL) and water (12 mL) is added over 30 minutes. The slurry was stirred for 3.5 hours, the filtered via Buchner funnel (Whatman #3 filterpaper). The white residue was washed twice with isopropanol (100 mL), then dried at 45° C. and 29 in for 19 hours to give the tartaric acid salt of the +enantiomer of N-phenyl carbanoyl eseroline tartrate (38.62 g) in 76% yield and 99.4% purity as a white solid.

### Example 4

The (+) enantiomer of formula I prepared in Example 2 was tested against its phenserine with respect to controlling β-APP levels in and the resulting toxic amyloid protein (Aβ protein) derived from the β-APP-protein by the procedure disclosed in the Shaw et al. Proc. Nat. Acad. Sci. USA (2001), 98 (13), 7605-7610. Pages 7506 and 7507 except that the test given below included (+) enantiomer of phenserine as well as phenserine itself so that phenserine and its (+) enantiomer were tested side by side for their effect in reducing the β-APP and Aβ-proteins. The methodology of Shaw et al for carrying out these tests is summarized as follows:

Drug treatment: SK-N-SH neuroblastoma cells were cultured on 60 mm dishes at a concentration of 3×106 cells, and SH-SY-5Y neuroblastoma cells were plated in 100 mm dishes at a concentration of 3×105 cells. The cells were allowed to grow in complete media (10% FBS, 2 mM glutamine in DMEM) for 3 to 4 days until they reached 70% confluence. To start the experiment, spent media were removed and replaced with fresh media (DMEM+0.5% FBS) containing 0, 5 or 50 μM of either (−)- or (+)-phenserine, and cells were incubated at 37 C, 5% CO₂ for the specific times indicated.

Lysate preparation: At each time point, the spent medium was collected and stored at −70 C for later analysis of secretory βAPP levels. Cell lysates were prepared as reported previously (Lahiri et al., 1997 and 1998). Protein levels of the supernatant were analyzed by the Bradford protein assay (BioRad, Mellville, N.Y.).

Western Blot: Fifteen μg of protein from each sample was laded onto a 10% NuPAGE Bis-Tris gel in 1× NuPAGE MOPS SDS running buffer (NOVEX, San Diego, Calif.) and the proteins separated at 200 V for 45 min. The gels then were transferred onto nitrocellulose at 25 V for 1.5 h. Non-specific binding was blocked, and each blot was probed for 2 h with either 22C11 anti-βAPP N-terminal antibody (2.5 μg/mL, Boehringer Mannheim, Indianapolis, Ind.) or anti-activated ERK antibody (25 ng/mL, Promega, Madison, Wis.). Anti-mouse Igg- or anti-rabbit IgG conjugated to horse radish peroxidase were used as secondary antibodies. Equivalent loading of samples was determined by Ponceau S staining (Sigma, St. Louis, Mo.). Densimetric quantification of the chemiluminesence of blots was undertaken by using a CD camera and NIH-IMAGE (version 4.1).

Lactate Dehydrogenase Assay: Measurement of released lactate dehydrogenase (LDH) in the conditioned medium was undertaken as a marker of cell viability and integrity, as described previously (Lahiri et al., 1997 and 1998)

Total Aβ Assay: Total Aβ peptide levels in SH-SY-5Y and SK-N-SH cultured samples were assayed by a sensitive ELISA (Suzuki et al., 1994). For total Aβ measurements, the a sandwich immunoassay with rabbit polyclonal antibody to residues 1-40 residues of Aβ as a capture antibody for all species of Aβ peptide Aβ1-40 and Aβ1-42 and the monoclonal antibody to 17-25 residues of Aβ was used to detect Aβ peptide levels, and the values were expressed as the mean of six independent assays.

Results

The results of this test are given in FIGS. 1 through 3. FIG. 1 demonstrates the decrease in βAPP levels can be proved to be control measured at various time intervals utilizing phenserine as various dosages of the 0.5 μM to 50 μM. As seen from FIG. 1 which is the same type of graph as the top figure of the second column on page 7507 of the Shaw, et al. publication, supra. FIG. 1 demonstrates that when compared to the control, the use of high dosages of phenserine decreased the βAPP levels in the SK-N-SH cells. In all cases even after 16 hours the amount of βAPP protein levels was reduced by the use of phenserine. FIG. 2 demonstrates that the levels of Aβ protein were substantially reduced from that of the controls especially after 10 hours through the use of phenserine. FIG. 3 compares the +enantiomer of phenserine with the (−) enantiomer of phenserine. As seen from this graph, both the negative and positive antipodes of phenserine are effective in reducing the βAPP levels as compared to the control as well as the levels of the Aβ protein from that of the control. Therefore (+)-phenserine antipode which lacks anticholinesterase activity has similar action on the β-APP and Aβ proteins as phenserine itself which is the (−) antipode in SK-N-SH cells.

### Example 5

Method In Vivo Studies

On administration of (−)-phenserine to rodents by the i.p. route (1 ml/kg in isotonic saline) a fine tremor is observed at a dose of 5 mg/kg. This is a classical central (i.e., brain) cholinergic over-stimulation (overdrive) effect. Such a tremor persisted for approximately 1 hour. Tremor, together with symptoms of peripheral over-stimulation (specifically, lacrimation and salivation) were seen at a dose of 7.5 mg/kg (−)-phenserine. At a dose of 20 mg/kg (−)-phenserine rodents are incapacitated by severe tremor and peripheral side effects (particularly salivation: making breathing difficult), and of 5 treated animals 2 were killed when moribund. However, when the same 20 mg/kg dose is given as (+)-phenserine, animals were entirely without symptoms (even minor tremor and appeared similar to both vehicle treated untreated animals).

Results: In Vivo Studies

(−)-Phenserine improves learning and performance in rodents (as well as in man), via its action as an anticholinesterase, to elevate levels of the cholinergic neurotransmitter, acetylcholine; which is depleted in the Alzheimer brain. The neurotransmitter, acetylcholine has numerous functions outside the brain, controlling heart rate (via the vagus nerve), gastric motility, sweating, salivation, lacrimation, etc. It is through stimulation of these actions, as well as over-stimulation of the brain cholinergic system, that results in the toxicity of classical anticholinesterases (e.g., the anticholinesterase drugs: rivastigmain and galanthamine as well as of phenserine at high doses. On the other hand, as seen from above (+)-Phenserine, however, lacks anticholinesterase activity and hence lacks cholinergic action. It can therefore be administered in higher amounts than (−)-phenserine.

### Example 6

A capsule is prepared utilizing the tartrate salt of the compound of formula I as the active ingredient (“The Active Ingredient”):

| | Amount per mg. |
|---|---|
| Active Ingredient | 50.0 |
| Microcrystalline cellulose NF (Avicel, PH₁₀₁) | 165.9 |
| Sodium starch glycolate NF (Primojel) | 9.0 |
| Net capsule fill weight approximately 260 mg. | |

### Example 7

1. Hard Gelatine Capsules Containing 100 mg, The Active Ingredient:

| Composition: One Capsule contains: | Amount per mg. |
|---|---|
| The Active Ingredient | 90.0 |
| Gelatine Bloom 30 | 70.0 |
| Maltodextrin MD₀₅ | 108.0 |
| dl-a-Tocopherol | 2.0 |
| Sodium ascorbate | 10.0 |
| Microcrystalline cellulose | 48.0 |
| Magnesium stearate | 2.0 |
| (weight capsule content) | 260.0 |

Procedure:

The Active Ingredient is wet milled in a solution of gelatine, Maltodextrin, dl-a-Tocopherol and sodium ascorbate.

The wet milled suspension is spray-dried

The spray-dried powder is mixed with microcrystalline cellulose and magnesium stearate.

260 mg. each of this mixture are filled into hard gelatine capsules of suitable size and color.

### Example 8

2. Table containing 150 mg. The Active Ingredient:

| Composition: | Amount per mg. |
|---|---|
| Tablet kernel: | |
| The Active Ingredient | 150.0 |
| Anhydrous lactose | 130.5 |
| Microcrystalline Cellulose | 80.0 |
| dl-a-Tocopherol | 2.0 |
| Sodium ascorbate | 10.0 |
| Polyvinylpyrrolidone K₃₀ | 5.0 |
| Magnesium stearate | 2.5 |
| (Kernel weight) | 250.0 |
| Film coat: | |
| Hydroxypropyl methylcellulose | 3.5 |
| Polyethylenglycol 6000 | 0.8 |
| Talc | 1.3 |
| Iron oxide, yellow | 0.8 |
| Titanium dioxide | 0.8 |
| (weight of the film) | 7.4 |

Procedure:

The active ingredient is mixed with anhydrous lactose and microcrystalline cellulose. The mixture is granulated in water with a solution/dispersion of Polyvinylpyrrolidone, dl-a-Tocopherol and sodium ascorbate.

The granular material is mixed with magnesium stearate and afterwards pressed as kernels wit h250 mg. weight.

The kernels are film coated with a solution/suspension of above-mentioned composition.

### Example 9

This example shows the means by which efficacy of the (+) enantiomer of formula I as a tartrate salt can be measured.

A randomized, double-blind, placebo-controlled study is done to measure the efficacy of the (+) phenserine tartrate or formation as in Example 6 in given daily over twelve (12) weeks, in 60 patients diagnosed as having symptoms similar to those caused by Alzheimer's disease (PAD). In this study there was a total of 60 eligible patients with PAD whose primary language is English, and the patients constituted male and female patients between the ages of 50 and 85 years.

Study Plan

Overall Study Design

General

Forty patients will receive two weeks of PT and a 50 mg BID dose level, at which time their dose will be escalated to 100 mg BID, where it will remain for the final ten (10) weeks. Concurrently, twenty patients assigned to placebo medication receive matched placebo capsules for the entire 12 week duration of the study. A sufficient number of potential patients are screened to ensure enrollment of 60 eligible cases.

All study participants were evaluated prior to the study (First Level) and periodically throughout using the following standard efficacy tests;

NPI (Neuropsychiatric Inventory,

CGIC (Clinician's Global Impression of Change)

ADAS-cog (Alzheimer's Disease Assessment Scale—cognitive subscale)

MMSE (Mini-Mental State Exam)

CANTAB (Cambridge Neuropsychological Test Automated Battery—

ADCS-ADL (Activities of Daily Living)

At the end of the twelve-week test, the patients in the treated group maintain a level at least as great as the First Level prior to treatment with respect to all of the above tests. In about 30% of the patients, there is an improvement in this level at the end of the twelve-week period. On the other hand, with respect to the untreated patients there was no improvement over the First Level as measured by the above tests and most of the patients in this control group show a decline from this First Level.

What is claimed:

1. A method for treating patients having disease state exhibiting cognitive impairments associated with aging or Alzheimer's disease which comprises administering to a patient having said cognitive impairments a composition containing an active ingredient selected from the group consisting of a compound of the formula: 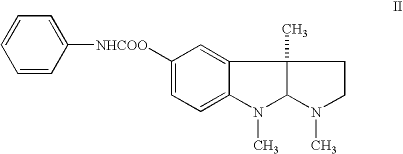
and its pharmaceutically acceptable salt,
said active ingredient and its salt being administered in an amount effective for retarding the progression of said disease states.

2. The method of claim 1 wherein said active ingredient is administered orally.

3. The method of claim 2 wherein said composition contains a pharmaceutically acceptable carrier.

4. A method for treating patients having disease state exhibits cognitive impairments associated with aging or Alzheimer's disease which comprises administering to a patient having said cognitive impairment a composition containing an active ingredient of the formula 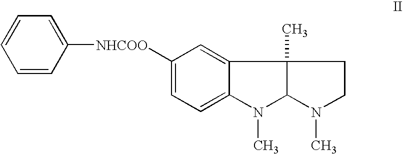
and salts thereof,
said composition being administered orally to provide the active ingredient to the patient a dose of from 0.5 mg to 10 mg/kg per day.

5. The method of claim 4 wherein said active ingredient is administered in an amount of from 1 to 5 mg/kg per day.

6. The method of claim 4 wherein said composition is in the form of a unit oral dosage form containing from 20 mg to 500 mg of the active ingredient.

7. A composition for treating patients having cognitive disorders comprising an active ingredient selected from the group consisting of a compound of the formula 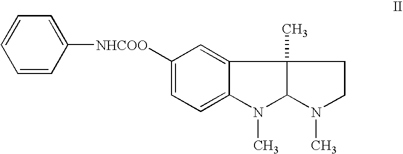
and its pharmaceutically acceptable salts,
and pharmaceutically acceptable carrier suitable for internal administration, said active ingredient being present in an amount suitable for retarding the progression of the disorder.

8. The composition of claim 7 wherein said composition contains said active ingredient in an amount sufficient to administer orally to a patient from about 0.5 to 10 mg/kg per day.

9. The composition of claim 8 wherein said active ingredient is contained in an amount sufficient to administer from about 1 to 5 mg/kg per day to a patient.

10. A composition in unit dosage form for oral administration comprising as an active ingredient a compound of the formula 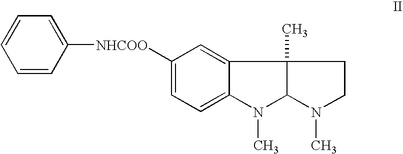
Or its pharmaceutically acceptable salts,
and a pharmaceutically acceptable carrier suitable for oral administration, said active ingredient being present in an amount of from about 20 to 300 mg.

11. The composition of claim 10 wherein said oral dosage form is a tablet or capsule.

12. The composition of claim 11 wherein said composition contains said active ingredient in an amount of from 50 mg to 200 mg.
